(19) United States
(12) Patent Application Publication
(10) Pub. No.: US 2025/0120887 A1
(43) Pub. Date: Apr. 17, 2025

(54) SKIN MOISTURIZING EXTERNAL COMPOSITIONS

(71) Applicant: Hongjong JIN, Sejong (KR)

(72) Inventors: Hongjong JIN, Sejong (KR); SeungKi HONG, Daejeon (KR)

(21) Appl. No.: 18/290,693

(22) PCT Filed: Sep. 14, 2023
(86) PCT No.: PCT/KR2023/013765

§ 371 Date: Jan. 19, 2024

(30) Foreign Application Priority Data
May 12, 2023 (KR) 10-2023-0062070

(51) Int. Cl. A61K 8/06; A61K 8/34; A61K 8/36; A61K 8/68; A61Q 19/00

(52) U.S. Cl. CPC A61K 8/062; A61K 8/342; A61K 8/361; A61K 8/68; A61Q 19/007; A61K 2800/591; A61K 2800/805

(57) ABSTRACT

The present invention relates to skin moisturizing external compositions containing a ceramide, a cholesterol, and a fatty acid, and more particularly, to skin moisturizing external compositions capable of preventing skin troubles such as atopy by forming a dense lamellar structure, which is an intercellular lipid structure, and improving the skin barrier, and may include a ceramide having long-chain fatty acids, a cholesterol, a long-chain fatty acid, and purified water as active ingredients.

### CROSS-REFERENCE TO RELATED APPLICATIONS

This application is the National Stage filing under 35 U.S.C. 371 of International Application No. PCT/KR2023/013765, filed on Sep. 14, 2023, which claims the benefit of Korean Patent Application No. 10-2023-0062070, filed on May 12, 2023, the contents of which are all hereby incorporated by reference herein in their entirety.

### TECHNICAL FIELD

The present invention relates to skin moisturizing external compositions containing ceramide, cholesterol, and fatty acid, and more particularly, to skin moisturizing external compositions capable of preventing skin troubles such as atopy by forming a dense lamellar structure, which is an intercellular lipid structure, and improving the skin barrier.

### BACKGROUND ART

The skin is largely composed of three layers: the subcutaneous layer, the dermal layer, and the epidermal layer. The epidermal layer is the outermost layer of the skin and serves to protect the skin. In the epidermal layer, the stratum corneum is the outermost layer of the epidermal layer, which serves to protect the skin from waste and irritation and acts as a protective film to prevent moisture evaporation.

In addition, the thickness and condition of the stratum corneum are related to the health of the skin. The stratum corneum is composed of keratinocytes and intercellular lipids. The keratinocytes are epidermal cells whose nuclei have disappeared and are stacked in layers in the keratin layer, so they not only serve as hydration for the keratin layer but also have resistance to ultraviolet rays, harmful substances, and physical stimulation.

The intercellular lipids are called Permeability barriers and are known to reduce moisture loss by suppressing evaporation of moisture in the body and function as antibacterial, antioxidant, and permeable barriers. The structure of the intercellular lipid is a lamellar structure in which the lipid double layer densely fills between the keratinocytes as an extracellular region of the keratin layer.

The components of the intercellular lipids are largely composed of ceramide, cholesterol, and free fatty acids. Human skin barrier lipid content consists of 50% ceramide, 25% cholesterol, and 15% fatty acids by weight, and exists in the form of a multi-lamellar lipid layer (lamellar membrane). And the above intercellular lipids control the permeation of substances, including moisture. Due to the composition and form of these intercellular lipids, cosmetic compositions combined with ceramide, cholesterol, and fatty acids are known to help improve skin barriers.

However, ceramide, cholesterol, and fatty acids, which are major intercellular lipid components, are not easily soluble in water, and even when combined in cosmetics, they are precipitated and crystal deposition. In particular, ceramide is a highly insoluble substance and has very low solubility in either aqueous or oil, making it difficult to use it as a cosmetic composition because the composition is not stably mixed, such as crystal deposition during distribution or storage, and even if dispersed through emulsification, it can be used in a very low content range.

Conventional skin moisturizing cosmetics stabilized with a high content of ceramide and a manufacturing method thereof are disclosed in Korean Patent Registration No. 10-1507883. However, the patent contains less than 1% by weight of ceramide, which limits its ability to provide compositions containing a high content of ceramide in a stable form.

In addition, since ceramides made of long-chain fatty acids, which are known to be insufficient for troubled skin such as atopy, have a larger molecular size of fatty acids than ceramides with short-chain fatty acids, it is more difficult to emulsify.

As a result, it is challenging to formulate a cosmetic composition containing ceramides with a long-chain fatty acid structure at a high concentration. Even when attempting to increase the ceramide content by reducing the size of emulsion particles for dispersion, the stability of ceramides is not significantly improved, and gelation occurs. This leads to precipitation issues during the formulation process, resulting in poor sensory attributes such as uneven application and poor spreadability. In the past, a solution to this problem involved manufacturing skin moisturizing external compositions containing a high concentration of ceramides using highly polar solvents. However, the use of such highly polar solvents adversely affects sensory attributes, making it difficult to incorporate them effectively as ingredients in cosmetic formulations.

### DISCLOSURE OF INVENTION

### Technical Problem

Accordingly, the present invention has been made to improve the conventional problems as described above, and an object of the present invention is to provide a skin moisturizing external composition with excellent skin compatibility by combining ceramides, cholesterol, and fatty acids—the major components of the skin's stratum corneum lipids—in a ratio to enhance the skin barrier.

In addition, another object of the present invention is to provide a skin moisturizing external composition that effectively helps to improve skin itching, improve skin dryness, and has excellent overall usability such as spreadability and absorbability.

In addition, a further object of the present invention is to provide a skin moisturizing external composition, wherein ceramides, including long-chain fatty acids that assist in improving the skin barrier and are beneficial for skin troubles like atopic dermatitis, are included at a high concentration without undergoing crystallization and precipitation, thereby ensuring the stability of the formulation.

The technical tasks that the present disclosure seeks to achieve are not limited to the challenges mentioned above. Any other unmentioned technical tasks will be clearly understandable to those skilled in the art from the disclosure below, in light of their common knowledge in the relevant technical field.

### Solution to Problem

In order to achieve the above technical challenges, a skin moisturizing external composition to an aspect of the present invention may include ceramides having long-chain fatty acids, cholesterol, long-chain fatty acids, and purified water as active ingredients.

In addition, the skin moisturizing external composition to an aspect of the present invention may include 2 to 16% by weight of the ceramide having a long-chain fatty acid, 1 to 8% by weight of the cholesterol, 1 to 8% by weight of the long-chain fatty acids and 30 to 60% by weight of the purified water.

In addition, in the skin moisturizing external composition to an aspect of the present invention, the ceramides having long-chain fatty acids are natural ceramides and may include one or more from the group consisting of ceramide NP, ceramide ENP, ceramide AP, ceramide EOP, ceramide NDS, ceramide ADS, ceramide EODS, ceramide NS, ceramide AS, ceramide EOS, ceramide NH, ceramide AH, ceramide EOH, and mixtures thereof.

In addition, in the skin moisturizing external composition to an aspect of the present invention, the ceramide having the long-chain fatty acid may include one or more from the group consisting of fatty acids having a chain length of 18 to 36 carbon atoms in the bound fatty acid.

In addition, in the skin moisturizing external composition to an aspect of the present invention, the cholesterol may include one or more from the group consisting of cholesterol, ergosterol, acaifamsterol, lanosterol, dihydrocholesterol, phytosterol, sitosterol, canolasterol, rice bran sterol, dolconsterol, tall oil sterol, rapeseed sterol, pomegranate sterol, beta-sitosterol, and a mixture thereof.

In addition, in the skin moisturizing external composition to an aspect of the present invention, the long-chain fatty acids may have a chain length of 14 to 22 carbons and may include one or more from the group consisting of palmitic acid, stearic acid, arachidic acid, behenic acid, linoleic acid, linolenic acid, arachidonic acid, and a mixture thereof.

In addition, the skin moisturizing external composition to an aspect of the present invention may further include a surfactant, fatty alcohols and oil.

In addition, in the skin moisturizing external composition to an aspect of the present invention, the surfactant may be a PEG FREE surfactant, and may include at least one from the group consisting of hydrogenatedreatitin, hydrogenated palm glyceride, sorbitan palmitate, sorbitan stearate, sorbitan isostearate, sorbitan sesquistearate, sorbitan oleate, sorbitan sesquioleate, sorbitan sesquioleate, sorbitan olivate, sucrose cocoate, methyl glucose sesquistearate, polyglyceryl-3 methyl glucosidase, methyl glucoside palmitate, glucoside palmitate, glucosetearyl, or glycolyl-3 methyl glucosidase At least one of decyl glucoside, coco glucoside, C12-20 alkyl glucoside (C12-20 alkyl glucoside), arachidyl glucoside, and a mixture thereof.

In addition, in the skin moisturizing external composition to an aspect of the present invention, the surfactant may be present in amounts ranging from 3.6 to 7.2% wt based on the total weight of the composition.

In addition, in the skin moisturizing external composition to an aspect of the present invention, the fatty alcohols may include at least one from the group consisting of cetyl alcohol, cetearyl alcohol, behenyl alcohol, cetanol, stearyl alcohol, oleyl alcohol, isostearyl alcohol, 2-octyldodecyl, 2-It may include at least one of ethylhexanocetyl, C14-22 alcohol, arachidyl alcohol, cetostearyl alcohol, batyl alcohol, and mixtures thereof.

In addition, in the skin moisturizing external composition to an aspect of the present invention, the fatty alcohols may be present in amounts ranging from 3.3 to 12 wt % based on the total weight of the composition.

In addition, in the skin moisturizing external composition to an aspect of the present invention, the oil may be a hydrocarbon-based oil, an ester-based oil, or a mixture thereof, and may include at least one or more from the group consisting of squalane, hydrogenated polydecene, acetylethylhexanoate, diglyceryl triisostearate, Capric/Caprylic Triglyceride, Glyceryltri 2-Ethylhexanoate, Isononylisononanoate, Ethylhexylisononanoate, Ethylhexyl Palmitate, Isostearyl Isostearate, Neopencil Glycol decafrate, Neopencil glycol diethylhexanoate, octyldodecyl myristate, pentaethrylytetraethylhexanoate, pentaethryly, and mixtures thereof.

In addition, in the skin moisturizing external composition to an aspect of the present invention, the oil may be present in amounts ranging from 9 to 19% wt based on the total weight of the composition.

In addition, the skin moisturizing external composition to an aspect of the present invention may further include polyols, polymers, and preservatives.

In addition, in the skin moisturizing external composition to an aspect of the present invention, the polyols may include at least one or more from the group consisting of glycerin, butylene glycol, dipropylene glycol, methyl propanediol, glycereth-26, diglycerin, propylene glycol, and may preferably include at least one of glycerin and dipropylene glycol and mixtures thereof.

In addition, in the skin moisturizing external composition to an aspect of the present invention, the polyols may be present in amounts ranging from 7 to 19% wt based on the total weight of the composition.

In addition, in the skin moisturizing external composition to an aspect of the present invention, the polymers may include at least one or more from the group consisting of carbomer, guar gum, xanthan gum, carboxymethyl cellulose, hydroxyethyl cellulose, sodium polyacrylate, hydroxypropyl cellulose, sodium hyaluronate, polyacrylic hydroxyethyl acrylate/sodium acryloyldimethyltaurate copolymer (hydroxyethyl acrylate/sodium acryloyldimethyltaurate copolymer), polyacrylate crosspolymer-6, ammonium, acryloyldimethyltaurate/Vpicopolymer, and may preferably include at least one or more from the group consisting of polyacrylate crosspolymer-6, hydroxyethyl acrylate/sodium acryloyldimethyl taurate copolymer (hydroxyethyl acrylate/sodium acryloyl dimethyl taurate copolymer) and mixtures thereof.

In addition, in the skin moisturizing external composition to an aspect of the present invention, the polymers may be present in amounts ranging from 0.2 to 2% wt based on the total weight of the composition.

### Advantageous Effects of Invention

According to the means for solving the above challenges, the skin moisturizing external composition according to the present invention is capable of excellent skin compatibility by mixing ceramide, cholesterol, and fatty acids in an effective ratio for skin absorption and having a structure like the lamellar structure of the skin.

Also, the skin moisturizing external composition according to the present invention can provide high content of ceramide having a long-chain fatty acid structure, which is insufficient for trouble skin such as atopy and the like, thereby rapidly restoring damaged skin barriers and deeply penetrating the skin to promote the differentiation. And thus, it can provide an excellent effect of relieving skin trouble such as atopy, psoriasis, and the like through the maintenance of skin homeostasis, improvement of moisturizing, and improvement of itching.

In addition, the skin moisturizing external composition according to the present invention can provide a stable form even when it contains a high content of ceramide having a long-chain fatty acid structure, which is an insoluble skin component, and can provide skin moisturizing usage with excellent application feeling such as spreadability and absorption.

The effects of the present invention are not limited to the above effects, and it should be understood to include all effects that can be inferred from the configuration of the invention described in the detailed description or the claims of the present invention.

### BRIEF DESCRIPTION OF DRAWINGS

FIGS. 1A and 1B illustrate schematic views of ceramide in the skin barrier, wherein FIG. 1A shows ceramides with short-chain fatty acids, and FIG. 1B shows Ceramides with long-chain fatty acids.

FIG. 2 is a photograph observing the properties of Example 1.

FIG. 3 is a photograph observing the properties of Comparative Example 1.

FIG. 4 is a photograph observing the properties of Comparative Example 2.

FIG. 5 is a graph showing a measurement result of skin moisture content of a product using the skin moisturizing external composition of the present disclosure.

FIG. 6 is a graph showing a measurement result of transdermal moisture loss of a product by using the skin moisturizing external composition of the present disclosure.

FIG. 7 is a graph showing the results of an itching (VAS) questionnaire evaluation of a product using the skin moisturizing external composition of the present disclosure.

FIG. 8 is a graph showing a satisfaction questionnaire evaluation result for a product using the skin moisturizing external composition of the present disclosure.

FIG. 9 is a graph showing a preference degree survey evaluation result for a product using the skin moisturizing external composition of the present disclosure.

### MODE FOR THE INVENTION

Hereinafter, the present invention will be described with reference to the accompanying drawings. However, the present invention may be implemented in various different forms and is not limited to an embodiment described herein.

Throughout the specification, when a certain portion is said to be “coupled (connected, contacted, or combined)” with another portion, it includes not only “directly coupled” but also “indirectly coupled” via another member. In addition, when a certain portion “includes” a certain component, it means that other components may be further provided, rather than excluding other components unless otherwise specified.

The terms used in the present specification are just used to describe a specific embodiment and are not intended to limit the present invention. Singular expressions include plural expressions unless clearly otherwise meant in context. In the present specification, terms such as “include” or “have” are intended to designate the presence of features, numbers, steps, actions, components, parts, or combinations thereof described in the specification and should be understood not to exclude in advance the possibility of the presence or addition of one or more other features, numbers, steps, actions, components, parts, or combinations

As used herein, the term “skin barrier improvement” refers to protection, reinforcement, or improvement of a skin barrier function of a stratum corneum. For example, the term “improvement of the skin barrier” refers to protection, reinforcement and/or improvement of the skin barrier function of the stratum corneum in the true sense through prevention of moisture reduction of the stratum corneum of the skin, for example, improvement of a moisture reduction index of the stratum corneum of the skin such as TEWL (percutaneous moisture loss), improvement of a skin barrier index such as an increase in skin hydration, an increase in production of ceramide, dihydroceramide and/or sphingo base, etc.

As used herein, the term “moisturizing” refers to maintaining homeostasis of a living body by properly controlling moisture loss (moisture evaporation) of skin. “Skin trouble” refers to a phenomenon in which the skin turns red due to external stimuli or physical changes, in some cases itching occurs, and in severe cases, small protrusions or pimple occur.

The present disclosure provides a skin moisturizing external composition comprising a ceramide having long-chain fatty acid, a cholesterol, and a long-chain fatty acid to improve a skin barrier.

Skin diseases accompanied by abnormalities in the skin barrier function include psoriasis, acute irritant dermatitis, acute occupational dermatitis, diaper dermatitis, allergic dermatitis, atopic dermatitis, chronic irritant dermatitis, chronic occupational dermatitis, psoriasis, hypertrophic scar, keloid scar, chillitis, lamellar ichthyosis, and the like. In addition, it has been found that abnormal skin barrier function is accompanied even in abnormal skin conditions such as sensitive skin or dry skin. Therefore, in various skin diseases or abnormal skin conditions as described above, the normalization of the skin barrier function or the enhancement of the skin barrier function is very important in the treatment of skin diseases or the normalization of skin functions.

The stratum corneum, the outermost layer of the skin, is a skin barrier composed of keratinocytes and intercellular lipids. The keratinocytes serve as a hydration for the stratum corneum and a barrier from ultraviolet rays and physical components or stimuli. The intercellular lipid is composed of ceramide, cholesterol, and free fatty acid, and has a lamellar structure in which a lipid bilayer tightly fills spaces between keratinocytes in an extracellular region of the keratinous layer. This lamella structure is a multi-layered structure in which hydrophilic and lipophilic layers are alternately and continuously stacked. The intercellular lipid is known to suppress the evaporation of moisture in the body due to the lamellar structure, thereby reducing loss and performing the function of an antibacterial and antioxidant permeable barrier.

In the case of dry skin and atopic skin, it is known that as the amount of ceramide in the intercellular lipid decreases, the lamellar liquid crystal structure in the skin is incompletely formed, and as a result, the loss of moisture in the skin increases, thereby causing abnormalities in the skin barrier function. In addition, ceramide- and ceramide-related compounds are known to serve as another important function in the skin, such as regulating signals related to the growth, differentiation, and death of cells in the skin.

Ceramide, which is a major component of the intercellular lipid, is the most abundant component which accounts for 50% of the intercellular lipid, and the lamellar structure is greatly influenced by ceramide. If ceramide is reduced, skin aging, wrinkles, and atopy may appear. Compositions which include high amounts of ceramide reduce the effects of aging, improve the radiancy and smoothness of the skin, and the like

The ceramide is a substance in which fatty acids are amide-bonded to a sphingolipid basic skeleton of sphingosine, phytosphingosine and sphinganin, and is classified into various ceramides according to the type of fatty acids and the type of sphingobases. In the skin, 7˜9 kinds of ceramides have been identified so far. Each ceramide group is composed of ceramides in which fatty acids of various carbon lengths are linked to amino groups of sphingoids. In the ceramides, the fatty acids bound to sphingolipids having ultra-long chain fatty acids such as C22, C24, etc., are presented for 40% or more, but other types of fatty acids having from C14 to C18 are variously distributed therein.

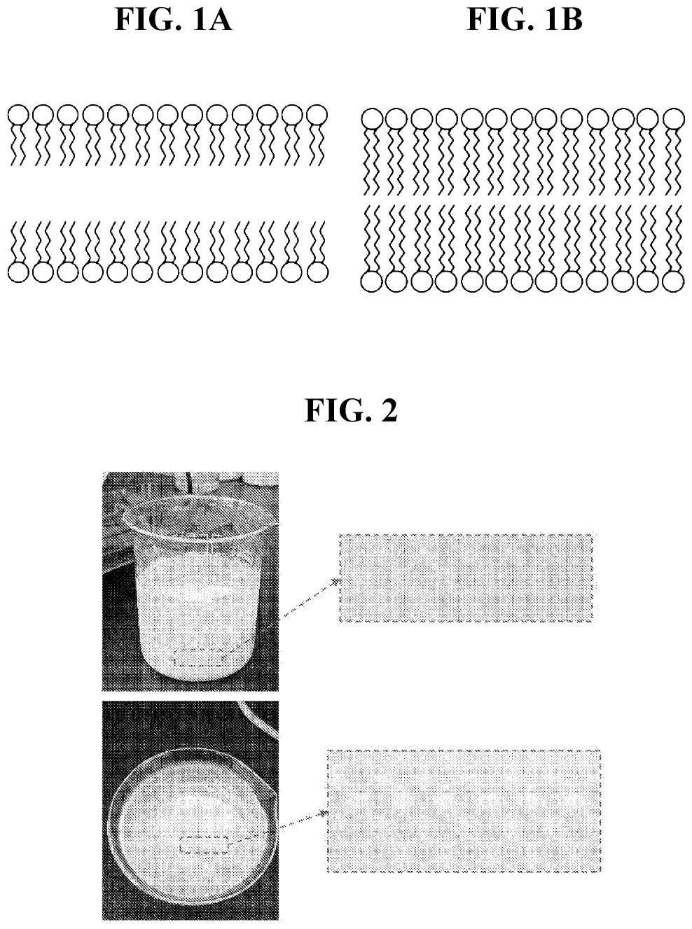

The ceramide surrounds keratinocytes, and when the length of the fatty acid chain constituting the ceramide is short as shown in (a) of FIG. 1, a wide gap is formed to surround the keratinocytes, and when the length of the fatty acid chain is long as shown in (b) of FIG. 1, there is no gap compared to the short-chain fatty acid, and thus tightly fills gaps between the keratinocytes to firmly support the keratinocytes.

Meanwhile, the distribution of fatty acids constituting the ceramides varies depending on the skin condition. When skin problems such as atopy occur, the length of the fatty acid chain is about 2 to 4 times shorter than that of healthy skin, and the distribution of the long chain fatty acids is relatively low. In particular, it is known that long- and very long-chain ceramides of C22, C24, and C26 tend to decrease in atopic skin. Also, healthy people can synthesize long-chain ceramide in the body by means of the activity of the enzyme that lengthens the length of the fatty acid chain. However, patients who have skin problems such as atopy, psoriasis, inflammation or the like have less enzyme activity for lengthening the length of the fatty acid, and thus it is difficult to synthesize long-chain ceramide in the body. Therefore, it is important to closely build the collapsed skin barrier by using ceramide and ceramide compounds that are rich in long-chain fatty acids, which are lacking in trouble skin such as atopy.

In the case of dry and sensitive troublesome skin with a damaged skin barrier, not only a decrease in ceramide but also abnormalities in the lipid feature itself are found. The intercellular lipid components are composed of 50% ceramide, 25% cholesterol, and 15% fatty acid, and maintaining the composition ratio of cholesterol and fatty acid as well as ceramide is a very important factor in the barrier homeostasis of the skin barrier. Therefore, to improve the skin barrier, it is known that the ceramide is more effective when used together with cholesterol and fatty acids constituting intercellular lipids than when used alone.

Accordingly, the external skin moisturizing externel composition of the present disclosure includes ceramide, cholesterol, and fatty acids as a feature, and the lipid mixture comprising ceramide, cholesterol, and fatty acid has a structure like that of skin lipid and penetrates deep into the skin, thereby quickly restoring damaged skin barriers.

The skin moisturizing external composition of the present disclosure may include a lipid mixture comprising ceramides having long-chain fatty acids, cholesterol, and long-chain fatty acid, which are like skin lipids, as an active ingredient for improving a skin barrier, and by further comprising PEG FREE surfactant, fatty alcohol, and oil in a predetermined ratio to the lipid mixture comprising the poorly soluble long-chain ceramide, has excellent emulsification stability and has low irritation to the skin.

The composition ratio of each component constituting the lipid mixture may be 2:1:1, 3:1:1, etc. for ceramides having long-chain fatty acids, cholesterol, and long-chain fatty acid, and the composition ratio is not limited thereto. The lipid mixture in which ceramide is mixed in the highest ratio among the feature components of the lipid mixture may be used.

The skin moisturizing external composition of the present disclosure may include ceramide, cholesterol, and fatty acid and purified water as active ingredients.

In the present disclosure, ceramide of the lipid mixture may include at least one of ceramide NP, ceramide ENP, ceramide AP, ceramide EOP, ceramide NDS, ceramide ADS, ceramide EODS, ceramide NS, ceramide AS, ceramide EOS, ceramide NH, ceramide AH, ceramide EOH, and a mixture thereof, and preferably, the ceramide NP or the ceramide ENP may be used alone or in a mixture thereof.

In addition, the fatty acids making up the ceramide may be selected from the group consisting of fatty acids having a carbon chain length of 14 to 36 carbon atoms, preferably, at least one fatty acid having a carbon chain length of 16 to 30 carbon atoms, and more preferably, at least one fatty acid having a carbon chain length of 16 to 26 carbon atoms.

According to an exemplary embodiment of the present disclosure, the amount of the ceramide may be 2 to 16 wt % with regard to the total weight of the skin moisturizing external composition.

The ceramide content less than 2 wt % fails to restore the skin barrier, and when it exceeds 16 wt %, there is a problem of poor emulsion stability, such as layer separation or crystal separation and precipitation.

In the present disclosure, the cholesterol of the lipid mixture may include at least one or more of cholesterol, ergosterol, acaifamsterol, lanosterol, dihydrocholesterol, phytosterol, sitosterol, canolasterol, rice bran sterol, dolconsterol, tall oil sterol, rapeseed sterol, pomegranate sterol, beta-sitosterol, and a mixture thereof, and more preferably, dolconsterol may be selected.

According to an exemplary embodiment of the present disclosure, the amount of cholesterol may be 1 to 8 wt % with regard to the total weight of the skin moisturizing external composition. The cholesterol content less than 1 wt % fails to restore the skin barrier, and when it exceeds 8 wt %, there is a problem of impair stability of the formulation.

In the present disclosure, the fatty acid may be selected at least one or more from the group consisting of long chain fatty acids, and preferably, a fatty acid comprising 14 to 22 carbon atoms, such as palmitic acid, stearic acid, arachidic acid, behenic acid, linoleic acid, linolenic acid, arachidonic acid, and a mixture thereof, and more preferably, stearic acid may be selected.

According to an exemplary embodiment of the present disclosure, the long chain fatty acid may be 1 to 8 wt % regarding the total weight of the skin moisturizing external composition. The long chain fatty acid content less than 1 wt % fails to restore the skin barrier, and when it exceeds 8 w %, there is a problem in that stability and usability of the formulation are impaired.

In the present disclosure, the amount of the purified water may be 30 to 60 wt % with regard to the total weight of the skin moisturizing external composition.

Although it contains a high content of poorly soluble ceramide, in order to have emulsion stability by uniformly dispersing it in the water phase, it may be mixed with PEG FREE surfactant, fatty alcohol, and oil at a certain ratio.

The skin moisturizing external composition of the present disclosure may include a surfactant, and the surfactant does not include polyethylene glycol (PEG). It is known that polyethylene glycol (PEG) has both hydrophilic and lipophilic properties so that inorganic powder is well positioned at the interface between water and oil, but PEG stimulates skin mucosa, causing skin troubles or urticaria. Therefore, the skin moisturizing external composition of the present disclosure is not irritating to the skin by using a surfactant not including PEG, and thus can be used for sensitive skin or trouble skin such as atopy.

In various embodiments, the skin moisturizing external composition may include one or more PEG FREE surfactant. Non-limiting examples of PEG FREE surfactant may be included, and preferably, it may include at least one from the group consisting of hydrogenatedreatitin, hydrogenated palm glyceride, sorbitan palmitate, sorbitan stearate, sorbitan isostearate, sorbitan sesquistearate, sorbitan oleate, sorbitan sesquioleate, sorbitan sesquioleate, sorbitan olivate, sucrose cocoate, methyl glucose sesquistearate, polyglyceryl-3 methyl glucosidase, methyl glucoside palmitate, glucoside palmitate, glucosetearyl, glycolyl-3 methyl glucosidase, decyl glucoside, coco glucoside, C12-20 alkyl glucoside, arachidyl glucoside, and a mixture thereof.

The amount of the PEG FREE surfactant may be 3.6 to 7.2 wt % with regard to the total weight of the skin moisturizing external composition, for example, 1 to 2 wt % of hydrogenated lecithin, 0.8 to 1.2 wt % of methylglucose sesquistearate, and 0.5 to 1 wt % of cetyl palmitate, 0.5 to 1 wt % of sorbitan olivate, 0.5 to 1 wt % of sorbitan palmitate, 0.2 to 0.5 wt % of C12-20 alkylglucoside, 0.1 to 0.5 wt % of arachidyl glucoside may be mixed and used. In the present disclosure, when the content of the PEG FREE surfactant is less than the above range, it is difficult to sufficiently emulsify the ceramide, thereby causing layer separation and precipitation, and when the content is more than the above range, stickiness and off-flavor may be severely generated, thereby deteriorating usability.

The skin moisturizing external composition of the present disclosure may include fatty alcohol as an emulsion stabilizer, and the fatty alcohol is an important component for forming liquid crystal emulsion and helps a lamellar structure at an interface, increases viscosity, and stabilizes a formulation. In addition, fatty alcohol has little irritation and helps to maintain moisture, protecting the skin.

The fatty alcohol may be included if they are generally used for topical application to the skin. Nonlimiting example of fatty alcohols may be included having 12 or more carbon atoms. Preferably, fatty alcohols may be chosen at least one from the group consisting of cetyl alcohol, cetearyl alcohol, behenyl alcohol, cetanol, stearyl alcohol, oleyl alcohol, isostearylalcohol, and 2-octyl alcohol, Dodecyl, 2-ethylhexanesetyl, C14-22 alcohol, arachidyl alcohol, batyl alcohol, and at least one of mixtures thereof, and more preferably behenyl alcohol, C14-22 alcohol, or arachidyl alcohol may be selected.

The amount of the fatty alcohols may be 3.3 to 12 wt % with regard to the total weight of the skin moisturizing external composition, and exemplary 1 to 6 wt % of cetearyl alcohol, 1 to 3 wt % of behenyl alcohol, 0.8 to 1.5 wt % of C14-22 alcohol, and 0.5 to 1.5 wt % of arachidyl alcohol may be mixed and used. In the present disclosure, when the content of the fatty alcohols is less than the above range, it is difficult to sufficiently emulsify the ceramide, thereby causing layer separation and precipitation, and when the content is more than the above range, formulation stability and usability may be deteriorated.

The skin moisturizing external composition of the present disclosure may include oil. The oil sufficiently dissolves ceramide and helps to form a strong interfacial layer to delay evaporation of moisture from the skin surface, and the oil commonly used in the art may be used.

Preferably, the oil may be selected from the group consisting of hydrocarbon oils and ester oils, and more preferably may be selected at least one or more from the group consisting of squalane, hydrogenated polydecene, acetylethylhexanoate, diglyceryltri Isostearate, Capric/Caprylic Triglyceride, Glyceryltri 2-Ethylhexanoate, Isononylisononanoate, Ethylhexylisononanoate, Ethylhexyl Palmitate, Isostearyl Isostearate, Neo Pencil Glycol Dicaprate, Neo Pencil Glycol Diethylhexanoate, Octyldodecyl Myristate, Pentaethryly Tetraethylhexanoate, Pentaethryly Tetraisostearate, Isotridensyl Isononanoate, Trimethylopropane Triisostearate, and mixtures thereof, and as an example, capric/caprylic triglyceride or squalane may be used alone or in combination.

Caprylic/capric triglyceride is a mixed triester of caprylic and capric acids and glycerin, which is made by reacting fatty acids present in palm oil with glycerin. It is known for its oxidative stability, which makes it a natural preservative, and its high penetrating power, which helps soften and smooth the skin.

Squalane is known to be a component that constitutes about 12% of the superficial fat of human skin, which has a high affinity and can reduce skin irritation and allergic reactions, and has a high absorption capacity due to its small particles, and has various functions such as moisturizing skin, inhibiting bacteria, and preventing skin aging. In addition, squalane has good miscibility with other ingredients and can be easily formulated, so it can be used in various formulations of cosmetics.

The amount of the oil may be 9 to 19 wt % with regard to the total weight of the skin moisturizing external composition, and preferably may be a mixture of 6 to 12 wt % capric/capric triglycerides and 3 to 7 wt % squalane. In the present disclosure, when the content of the oil is less than the above range, it is difficult to sufficiently dissolve the ceramide, when the content of the oil is exceeded above range, it is difficult for the oil phase components to effectively form the emulsified particles, and thus dispersion of the formulation and long-term stability may be deteriorated.

The skin moisturizing external composition according to an embodiment of the present disclosure may further include polyols, and the polyols in order to serve to diversify the formulation by improving the stability of ceramides and suppressing the occurrence of gelation or precipitation over time.

The polyol may be one or more selected from the group consisting of glycerin, butylene glycol, dipropylene glycol, methyl propanediol, glycerol-26, diglycerin, propylene glycol, and mixtures thereof, and preferably, may be one or more selected from glycerin, dipropylene glycol, and a mixture thereof.

The amount of the polyol may be 7 to 19 wt % with regard to the total weight of the skin moisturizing external composition, and preferably may be a mixture of 4 to 9 wt % glycerin and 3 to 10 wt % dipropylene glycol.

When the content of the polyol is less than the above range, it is difficult for ceramide to be uniformly dispersed, and when the content of the oil is exceeded the above range, there is a problem in that the formulation becomes unstable.

The skin moisturizing external composition according to an embodiment of the present disclosure may further include a thickener, preservative, raw material by-product, and a metal ion sequestering agent.

The skin moisturizing external composition of the present disclosure may include a polymer for controlling viscosity as a thickener, wherein it is one of the components contained in the aqueous phase part. The polymer may be selected from the group consisting of carbomer, guar gum, xanthan gum, carboxy methyl cellulose, hydroxyethyl cellulose, sodium polyacrylate, hydroxypropyl cellulose and sodium hyaluronate, polyacrylate, polyacrylamide, hydroxyethylacrylate/sodium acryloyldimethyltaurate copolymer, polyacrylate crosspolymer-6, ammonium acryloyldimethyltaurate/V-picopolymer, and a mixture thereof, and preferably may be selected at least one of polyacrylate crosspolymer-6, hydroxyethylacrylate/sodium acryloyldimethyltaurate copolymer and a mixture thereof.

In the present disclosure, the polymer included may preferably range from 0.2 to 2 wt %, and for example, 0.1 to 1 wt % of polyacrylate crosspolymer-6, and 0.1 to 1 wt % of hydroxyethylacrylate/sodium acryloyldimethyltaurate copolymer may be mixed and used. In the present disclosure, the polymer content less than 0.2 wt % fails to form viscosity, and when the content of the polymer exceed 2.0 wt %, formulation are deteriorated due to an increase in viscosity.

The skin moisturizing external composition of the present disclosure may further include 1 to 5 wt % of 1,2-Hexanediol for preservative purposes, and may include 0.01 to 1 wt % of Ethylhexylglycerin as a preservative aid.

Herein, 1,2-Hexanediol can be included as a preservative or antibacterial raw material for long-term safe use of external compositions or cosmetics, and, it has the function of helping to improve the preservative power of external compositions or cosmetics by easily destroying microbial cell membranes, and in addition, it has moisturizing and solvent functions and can be used for various purposes.

Ethylhexylglycerin, a type of glycerin, is a multifunctional ingredient that can be used as a deodorant or skin conditioning agent in addition to its antibacterial function. In addition, it has a hydrophilic-lipophilic balance (HLB) of 7.4, which indicates the degree of lipophilic and hydrophilic groups, so it reduces the surface tension of microbial cell membranes and helps other antibacterial agents to contact the cell membrane, providing improved effects when used in combination with other antibacterial agents.

The skin moisturizing external composition according to an embodiment of the present disclosure may further include sorbitan isostearate as a raw material by-product having emulsifying capabilities. In the present disclosure, sorbitan isostearate included may range from of 0.001 to 0.01 wt %.

The method for preparing the skin moisturizing external composition according to the present disclosure is not particularly limited, and a conventional method for preparing an oil-in-water type external composition may be used.

The method for producing the skin moisturizing external composition of the present disclosure may be exemplarily comprised as follows.
1) Prepare an aqueous phase part by heating to 55 to 85° C., wherein the aqueous phase part contains purified water, and, as the polyol, glycerin and dipropylene glycol.
2) Prepare an oil phase part containing ceramides, cholesterols, long-chain fatty acids, oils, fatty alcohols, and surfactants by heating to 65 to 85° C.
3) Inject the oil phase into the aqueous phase to mix and add polymers and preservatives. The mixing may be accomplished using a homo mixer, and the speed and time of stirring may vary depending on the ingredients used, preferably stirring is performed at RPM 3000 to 7000 for 15 to 35 minutes.
4) Cool to prepare the skin moisturizing external composition of the present disclosure.

The skin moisturizing external composition may be used for the actual usage in the form of any one of solution, external ointment, cream, liquid, foam, nourishing lotion, soft lotion, pack, soft water, body wash, emulsion, makeup base, essence, soap, liquid detergent, bath preparation, sun screen cream, sun oil, suspension, emulsion, paste, gel, lotion, powder, soap, foam cleansing, oil, powder foundation, emulsion foundation, wax foundation, patch, and spray.

### Example 1

A skin moisturizing external composition were prepared in compositions shown in Table 1 below. The ceramide used in Example 1 is “HP EcoCeramide LCS” (Ceramide NP, C16˜C24) from LC Biotech (https://www.lcsbio.com).

The skin moisturizing external composition of Example 1 was produced by the preparation method of the skin moisturizing external composition of the present disclosure.

In detail, each material was measured in a beaker according to each component and content listed in Table 1, the aqueous phase part was heated to 55° C.˜85° C., the oil phase part was heated to 65° C.˜85° C. to dissolve, and then the oil phase part was slowly added to the aqueous phase part. At this time, the mixing of the aqueous phase portion and the oil phase portion was stirred at a speed of 3000 to 7000 RPM for 15 to 35 minutes using a homogenizer, and then cooled to prepare the skin moisturizing external composition of Example 1.

### Comparative Examples 1 and 2

In Comparative Examples 1 and 2, the compositions of ceramide, cholesterol, fatty acid, and purified water were the same as that of Example 1, and contents of surfactant, fatty alcohol, and oil for emulsifying the lipid mixture were different from those of Example 1 as shown in Table 1 below, and polyol, polymer, preservatives, and other components were prepared in the same compositions as that of Example 1.

The manufacturing methods of Comparative Examples 1 and 2 were used in the same method as in Example 1.

TABLE 1

| | | Unit: wt % | | |
|---|---|---|---|---|
| | | | Compar- | Compar- |
| | | | ative | ative |
| ingredient | | Example 1 | Example 1 | Example 2 |
| Oil-phase | ceramide | 5.87 | 5.87 | 5.87 |
| part | cholesterol | 2.74 | 2.74 | 2.74 |
| | fatty acid | 3.33 | 3.33 | 3.33 |
| | Surfactants | 5.68 | 2.54 | 5.68 |
| | fatty alcohols | 5.28 | 5.28 | 1.76 |
| | oil | 17.22 | 21.33 | 21.33 |
| Aqueous- | Purified water | 48.34 | 48.34 | 48.34 |
| phase part | | | | |

### Experimental Example 1: Confirmation of Formulation Stability

The skin moisturizing external composition of Example 1 was stably emulsified by densely and uniformly dispersing the particles in the water phase, and as shown in FIG. 2, there was no layer separation or precipitation, and it showed a suitable appearance as a cream.

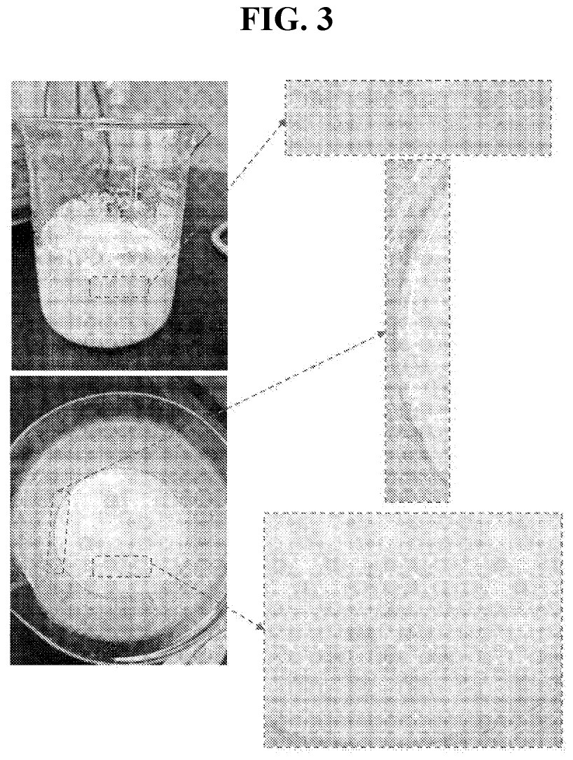

However, in Comparative Example 1, where the content of the surfactant was set to be 2.54 wt % and the content thereof was lower than the range of the surfactant suggested in the present disclosure, even though the content of the oil was increased to be 21.33 wt % by supplementing the oil, since the content of the surfactant was low, thus particles were not uniformly dispersed in the water phase of the composition and emulsion stability was decreased, and as shown in FIG. 3, a layer separation phenomenon and crystals were precipitated.

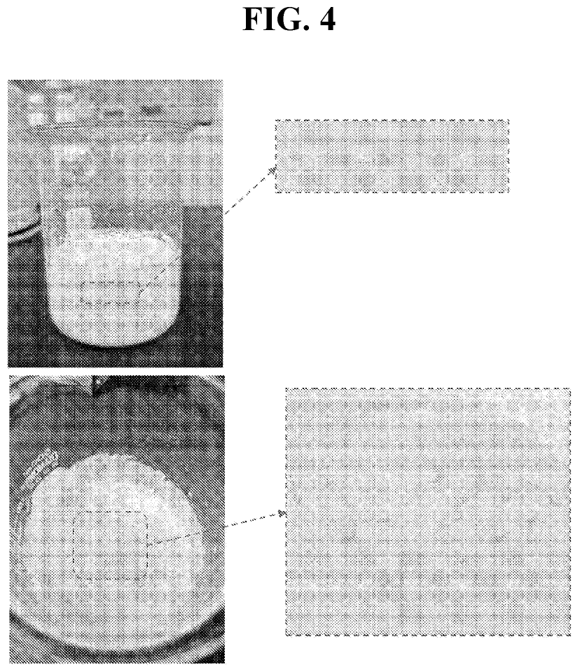

In addition, in Comparative Example 2, where the content of the fatty alcohol was 1.76 wt % and was lower than the range of the fatty alcohol suggested in the present disclosure, as in Comparative Example 1, even though the oil was increased to 21.33 wt % by supplementing the oil, since the content of the fatty alcohol was low, thus the emulsion stability was low, such that the layer separation phenomenon and the crystals were precipitated as shown in FIG. 4.

The skin moisturizing external composition of the present disclosure can obtain emulsion stabilization without separation of properties or precipitation of crystals through easy mixing of poorly soluble skin lipid components, which are difficult to emulsify, with surfactants, fatty alcohols, and oils.

### Experimental Example 2: Confirmation of Skin Hydration Improvement Effect

The moisturizing effect, the itching alleviating effect, and the stability of the skin moisturizing external composition of the present disclosure will be described through a human body application test of a clinical trial product including the skin moisturizing external composition of Example 1 according to the present disclosure.

The clinical trial product was prepared by including 54.50 wt % of Example 1, and 45.50 wt % of an additional material based on the total weight thereof. The additional material includes 6.8 wt % of Centella Asiatica Extract, 37 wt % of purified water, 0.3 wt % of a thickener, and 1.4 wt % of a concept material (panthenol Panthenol, Tremella Fuciformis (Mushroom) Extract of Tremella foliacea).

Subjects were 22 (45.9±8.71 years old), with itchy skin but no skin diseases or skin allergies, who applied the product to their forearms for 2 weeks. The subject has a transepidermal water loss of 12.0 g/m²/h or more, and the subjects' test areas were kept clean and dry, and the test was conducted under the same measurement conditions: constant temperature and humidity (room temperature 20-24° C., humidity 40-60%) with no air movement and no direct sunlight.

### 2-1 Measurement of Skin Hydration

The skin hydration level of the forearm was measured using a skin moisture meter (Corneometer, CM825, Courage+Khazaka Electronic GmbH) and evaluated before and after product use.

As a result, as shown in the table 2 below, it was measured as 31.48 A. U before using the product, and 53.25 A.U after two weeks of using the product, and it showed a statistically significant increase in skin hydration level after two weeks of using the product compared to before using the product. (p<0.05)

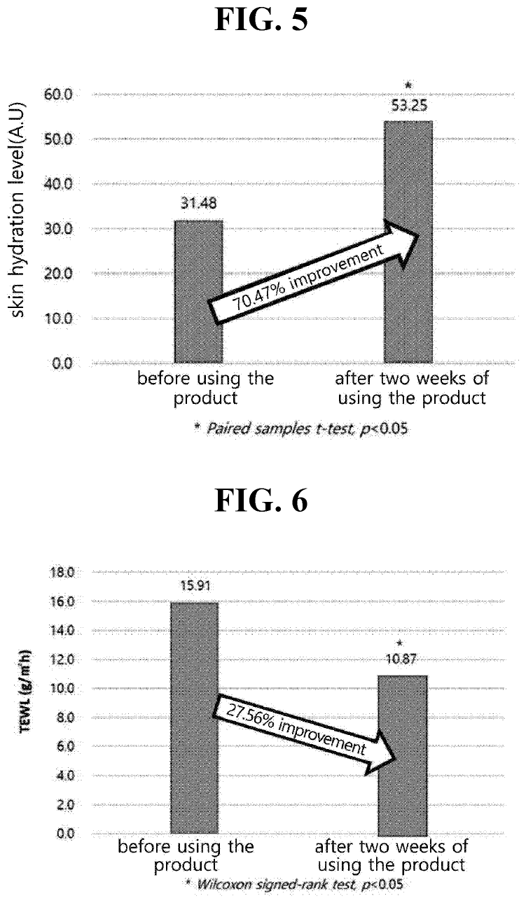

According to the improvement analysis of skin hydration, there is an improvement of 70.47% after 2 weeks of using the product compared to before using the product, as shown in Table 2 and the graph in FIG. 5.

TABLE 2

| | Measures | Improvement | |
|---|---|---|---|
| | (mean ± | rate | |
| Viewpoint | standard error) | (%) | p-value |
| Before using the | 31.48 ± 4.209 | — | |
| product | | | |
| After 2 weeks of | 53.25 ± 9.472 | 70.47 ± 30.392 | 0.000 |
| product use | | | |

(unit: g/m² h )

### 2-2 Measurement of Trans-Epidermal Water Loss (TEWL)

The transepidermal water loss of the forearm was measured and analyzed using Tewameter TM300 (Courage+Khazaka Electronic GmbH) and evaluated before using the product. It was measured at 15.91 g/m2-h before product use, and 10.87 g/m2-h after 2 weeks of product use, indicating a statistically significant reduction in transepidermal water loss after 2 weeks of product use compared to before product use (p<0.05). The results of the transdermal water improvement rate analysis showed an improvement of 27.56% after 2 weeks of using the product compared to before using the product, as shown in the graph in FIG. 6, and summarized in Table 3 below.

TABLE 3

| | Measures | Improvement | |
|---|---|---|---|
| | (mean ± | rate | |
| Viewpoint | standard error) | (%) | p-value |
| Before using the | 15.91 ± 6.223 | — | |
| product | | | |
| After 2 weeks of | 10.87 ± 1.482 | 27.56 ± 13.958 | 0.000 |
| product use | | | |

(unit: g/m² h)

### 2-3 Itching Survey (VAS)

The degree of itching felt by the test subjects was measured using the questionnaire evaluation. In the evaluation, the degree of skin itching (Score) before and after two weeks of product use was self-evaluated, and compared with before product use. The subjects evaluated the 10-point scale for maximum itching at the time of visit with an itching evaluation tool, visual analogue scale (VAS). The subject indicated the degree of itching directly on the 10 cm line segment and the degree was evaluated. The itching scale is as shown in Table 3 below, and it means that the itching is improved when the VAS score is lowered by evaluating the itching improvement degree by comparing the degrees indicated by the subjects.

TABLE 4

| score | degree of itching |
|---|---|
| 0 points | no itching |
| More than 1 point and less | mild itching |
| than 3 points | (milde pruritus) |
| 3 or more and less than 7 | Moderate itching (moderato pruritus) |
| 7 or more and less than 9 | severe itching (severe pruritus) |
| 10 points | very severe pruritus |

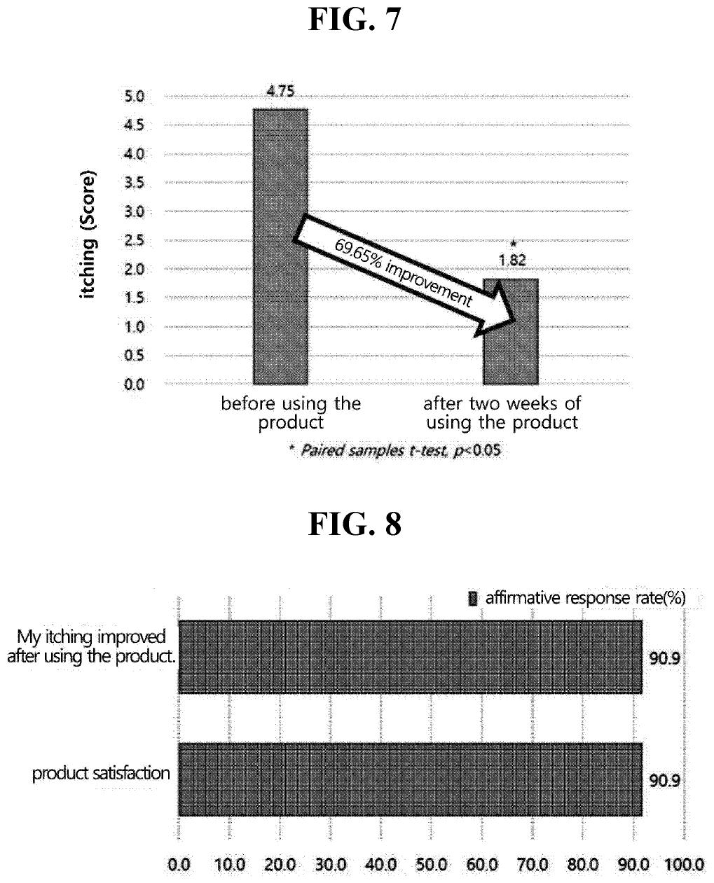

As a result of the itching questionnaire evaluation, it was evaluated as 4.75 score before using the product and 1.82 score two weeks after using the product, and showed statistically significant itching alleviation two weeks after using the product compared to before using the product (p<0.05). The itch improvement rate analysis showed a 69.65% improvement rate after 2 weeks of using the product compared to before using the product, as shown in the graph in FIG. 7, and shown in Table 5 below.

TABLE 5

| | Measures | Improvement | |
|---|---|---|---|
| | (mean ± | rate | |
| point of view | standard error) | (%) | p-value |
| Before using the | 4.75 ± 1.631 | — | |
| product | | | |
| After 2 weeks of | 1.82 ± 1.842 | 69.65 ± 36.396 | 0.000 |
| product use | | | |

(Unit: score)

### 2-4 Stability Evaluation

It was confirmed whether there was an adverse reaction immediately after using the product for each subject. For the evaluation of the adverse reaction, it was closely observed by eye whether the adverse reaction (erythema, edema, perforation, itching, self-pain, burning, stiffness, tingling and other adverse reactions) occurred every time the subject visited. As a result, all 22 subjects answered that there were no symptoms.

### 2-5 Product Satisfaction and Preference Survey Rating

The subjects completed a subjective satisfaction rating and a product preference rating after using the test product.

Satisfaction ratings were based on “My itching improved after using the product.” and “product satisfaction” into 5 points with very good, 4 points with good, 3 points with neutral, 2 points with poor, and 1 point with very poor, and the results were summed and expressed as a percentage.

As a result of the analysis of the product satisfaction questionnaire, as shown in FIG. 8, 90.9% of the respondents affirmed that “My itching improved after using the product.”, and 90.9% of the respondents affirmed that “product satisfaction” indicates that itching is improved and satisfaction with the product is high.

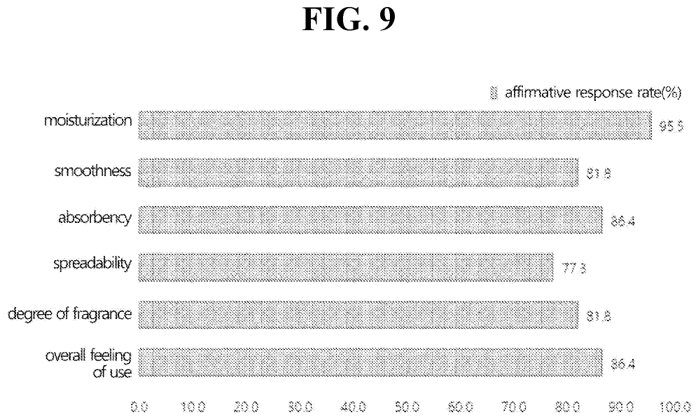

Preference rating was performed for the feeling of moisturization, smoothness, absorbency, spreadability, degree of fragrance, and overall feeling of use with 5 points of very good, 4 points of good, 3 points of neutral, 2 points of poor, and 1 point of very poor, and the results were summed and expressed as a percentage. As a result of analyzing the preference questionnaire, as shown in FIG. 9, it was found that 95.5% of the moisturization, 81.8% of the smoothness, 86.4% of the absorbency, 77.3% of the spreadability, 81.8% of the degree of fragrance, and 86.4% of the overall feeling of use.

### 3 Evaluation of Primary Skin Irritation

Next, evaluation of skin irritation of the skin moisturizing external composition of the present disclosure will be described through a human skin primary irritation test of the skin moisturizing external composition prepared according to Example 1 of the present disclosure.

There were 30 subjects, aged 20-60 years, and the test method was to instill 20 ul of the test product into the IQ Ultra Patch, apply it to the back, fix it with 3M Micropore Tape, rating the skin irritation index, and take pictures. The first determination was made 1 hour after patch removal in the past 24 hours of patch attachment, and the second determination was made 24 hours after patch removal. The skin irritation was visually assessed according to the Frosch & Kligman and CTFA guidelines and the International Contact Dermatitis Research Group (ICDRG) criteria.

### <Skin Determination Standard>

(−): 0.0—no irritation at all
(±): 0.5—very weak redness
(+): 1—weak redness
(++): 2—Intense flare
(+++): 3—Very strong redness or edema

### <Final Judgment Standard>

0.0-0.9: low irritation, 1.0-2.9: light irritation, 3.0-4.9: medium irritation, 5.0 or more: strong irritation

TABLE 6

| | | | | | | | | | Average skin | |
|---|---|---|---|---|---|---|---|---|---|---|
| | Primary read | | | | Secondary read | | | | response degree | Judgment |
| Number | ± | 1+ | 2+ | 3+ | ± | 1+ | 2+ | 3+ | | |
| of reactant | | | | | | | | | | |
| 0 | 0 | 0 | 0 | 0 | 0 | 0 | 0 | 0 | 0.00 | Low (no) |
| | | | | | | | | | | irritation 0 |
| | | | | | | | | | | 0 |

As a result of the primary skin irritation test, the skin moisturizing external composition according to an embodiment of the present disclosure had an average skin response of 0.00, which was determined as low (no) irritation.

In addition to this, the foregoing description of the present invention is for illustrative purposes only, and those having ordinary knowledge in the art to which the present invention pertains will appreciate that the present invention can be easily modified in other specific forms without changing the technical spirit or essential features. Therefore, it should be understood that the embodiment described above is exemplary and non-limiting in all respects. For example, each component described as a single type may be carried out as being distributed or divided, and similarly, components described as being distributed or divided may also be carried out in a combined form within the scope of the understanding of those having ordinary skill. In addition, the steps of the method may be carried out in such a manner that each step is performed multiple times or is performed multiple times in combination with at least one other step.

It should be interpreted that the scope of the present invention is represented by the claims to be described below, and all changed or modified forms derived from the meaning and scope of the claims and the equivalent concept thereof are included in the scope of the present invention.

This research was supported by “Regional Innovation Strategy (RIS)” through the National Research Foundation of Korea (NRF) funded by the Ministry of Education (MOE) (2021RIS-001).

1. A skin moisturizing external composition comprising: a ceramide having long-chain fatty acids; a cholesterol; a long-chain fatty acid; and purified water.

2. The skin moisturizing external composition according to claim 1,
wherein the composition comprises 2 to 16 wt % of the ceramide with long-chain fatty acids, 1 to 8 wt % of the cholesterol, 1 to 8 wt % of the long-chain fatty acid, and 30 to 60 wt % of purified water.

3. The skin moisturizing external composition according to claim 1,
wherein the ceramide having long-chain fatty acids comprises at least one of ceramide NP, ceramide ENP, ceramide AP, ceramide EOP, ceramide NDS, ceramide ADS, ceramide EODS, ceramide NS, ceramide AS, ceramide EOS, ceramide NH, ceramide AH, ceramide EOH, and mixtures thereof.

4. The skin moisturizing external composition according to claim 1,
wherein the ceramide having long-chain fatty acids comprises one or more from the group consisting of fatty acids having a chain length of 14 to 36 carbon atoms; or preferably comprises at least one from fatty acids having a chain length of 16 to 30 carbon atoms; or more preferably comprises at least one from fatty acids having a chain length of 16 to 26 carbon atoms.

5. The skin moisturizing external composition according to claim 1,
wherein the cholesterol comprises at least one or more from the group consisting of cholesterol, ergosterol, acaifamsterol, lanosterol, dihydrocholesterol, phytosterol, sitosterol, canolasterol, rice bran sterol, dolconsterol, tall oil sterol, rapeseed sterol, pomegranate sterol, beta-sitosterol, and a mixture thereof.

6. The skin moisturizing external composition according to claim 1,
wherein the long-chain fatty acid comprises at least one or more from the group consisting of acid comprising 14 to 22 carbon atoms, and comprises at least one or more from the group consisting of palmitic acid, stearic acid, arachidic acid, behenic acid, linoleic acid, linolenic acid, arachidonic acid, and a mixture thereof.

7. The skin moisturizing external composition according to claim 1,
further comprising a surfactant, a fatty alcohol and an oil.

8. The skin moisturizing external composition according to claim 7,
wherein the surfactant is a PEG FREE surfactant, and comprises at least one from the group consisting of hydrogenated lecithin, hydrogenated palm glyceride, sorbitan palmitate, sorbitan stearate, and sorbitan isostearate (Sorbitan isostearate), sorbitan sesquistearate (Sorbitan sesquistearate), sorbitan oleate (Sorbitan oleate), sorbitan sesquioleate (Sorbitan sesquioleate), sorbitan Olivate (Sorbitan olivate), sucrose cocoate (Sucrose cocoate), methyl glucose sesquistearate (methyl glucose sesquistearate), polyglyceryl-3 methylglucose distearate (Polyglyceryl-3 Methylglucose Distearate), cetyl palmitate (Cetyl Palmitate), Cetearyl Glucoside, Decyl Glucoside, Coco Glucoside, C12-20 Alkyl Glucoside, Arachidyl Glucoside (Arachidyl Glucoside) and a mixtures thereof.

9. The skin moisturizing external composition according to claim 7,
wherein the surfactant comprises from 3.6 to 7.2 wt % based on the total weight of the composition.

10. The skin moisturizing external composition according to claim 7,
wherein the fatty alcohol comprises at least one from the group consisting of cetyl alcohol, cetearyl alcohol, behenyl alcohol, cetanol, stearyl alcohol, oleyl alcohol, isostearyl alcohol, 2-octyldodecyl, 2-ethylhexanocetyl, C14-22 alcohol, arachidyl alcohol, batyl alcohol and mixtures thereof.

11. The skin moisturizing external composition according to claim 7,
Wherein the fatty alcohol comprises from 3.3 to 12 wt % based on the total weight of the composition.

12. The skin moisturizing external composition according to claim 7,
wherein the oil comprises hydrocarbon oils or ester oils, and comprises at least one from the group consisting of squalane, hydrogenated polydecene, acetylethylhexanoate, diglyceryltriisostearate, capric/caprylic triglyceride, Glyceryl Tri-2-ethylhexanoate, isononylisononanoate, ethylhexylisononanoate, ethylhexyl palmitate, isostearyl isostearate, neopencil glycol dicaprate, neopencil glycol diethylhexanoate, Octyldodecyl myristate, pentaethryly tetraethylhexanoate, pentaethryly, and mixtures thereof.

13. The skin moisturizing external composition according to claim 7,
wherein the oil comprises from 9 to 19 wt % based on the total weight of the composition.

14. The skin moisturizing external composition according to claim 7,
further comprising a polyol, a polymer, and a preservative.

15. The skin moisturizing external composition according to claim 14,
wherein the polyol comprises one or more from the group consisting of glycerin, butylene glycol, dipropylene glycol, methyl propanediol, glycereth-26, diglycerin, propylene glycol and mixtures thereof.

16. The skin moisturizing external composition according to claim 14,
wherein the polyol comprises from 7 to 19 wt % based on the total weight of the composition.

17. The skin moisturizing external composition according to claim 14,
wherein the polymer comprises at least one from the group consisting of carbomer, guar gum, xanthan gum, carboxy methyl cellulose, hydroxyethyl cellulose, sodium polyacrylate, hydroxypropyl cellulose and sodium hyaluronate, polyacrylate, polyacrylamide, hydroxyethyl Acrylates/Sodium Acryloyl Dimethyl Taurate Copolymer, Polyacrylate Crosspolymer-6, Ammonium, Acryloyl Dimethyl Taurate/Vpico and mixtures thereof.

18. The skin moisturizing external composition according to claim 14,
wherein the polymer comprises from 0.2 to 2 wt % based on the total weight of the composition.
